(19) United States
(12) Patent Application Publication
(10) Pub. No.: US 2021/0096099 A1
(43) Pub. Date: Apr. 1, 2021

(54) ANALYSIS CHIP

(71) Applicant: Aipore Inc., Tokyo (JP)

(72) Inventors: Kentaro Kobayashi, Tokyo (JP); Hiroshi Hamasaki, Hiratsuka Kanagawa (JP); Ping Wang, Kawasaki Kanagawa (JP); Naofumi Nakamura, Tokyo (JP)

(21) Appl. No.: 17/113,710

(22) Filed: Dec. 7, 2020

Related U.S. Application Data
(62) Division of application No. 15/461,891, filed on Mar. 17, 2017.

(30) Foreign Application Priority Data
Sep. 23, 2016 (JP) 2016-185493

(51) Int. Cl. G01N 27/447; B01L 3/00; G01N 15/12; C12Q 1/04

(52) U.S. Cl. CPC G01N 27/44791; B01L 3/502761; G01N 2015/1006; C12Q 1/04; G01N 15/1245

(57) ABSTRACT

According to one embodiment, an analysis chip for detection of particles in a sample liquid includes a substrate, a channel provided on a surface portion of the substrate, a liquid storage portion provided on a part of the channel to store the sample liquid, holes being provided at a bottom portion of the liquid storage portion to connect the liquid storage portion and the channel, and first electrodes provided in the channel or the liquid storage portion.

### CROSS-REFERENCE TO RELATED APPLICATIONS

This application is a divisional of application Ser. No. 15/461,891 filed on Mar. 17, 2017 and is based upon and claims the benefit of priority from Japanese Patent Application No. 2016-185493, filed Sep. 23, 2016, the entire contents of which are incorporated herein by reference.

### FIELD

Embodiments described herein relate generally to an analysis chip for detection of particles in a sample liquid.

### BACKGROUND

Recently, in the field of biotechnology and healthcare, attention has been focused on semiconductor micro-analysis chips on which microfluidic elements such as micro flow channels and detection mechanisms are integrated. The analysis chip of this type can detect particles and biopolymers contained in the sample liquid flowing in a micro flow channel by means of measuring an electrical signal change which occurs when the particles in the sample liquid pass through micropores formed in the flow channel.

### BRIEF DESCRIPTION OF THE DRAWINGS

FIG. 1 is a perspective view showing a schematic structure of a semiconductor micro-analysis chip of a first embodiment.

FIG. 2 is a cross-sectional view showing section I-I′ in FIG. 1.

FIG. 3 is a cross-sectional view for explanation of a method of inspecting particles.

FIGS. 4A to 4I are cross-sectional views showing steps of manufacturing the semiconductor micro-analysis chip of the first embodiment.

FIG. 5 is a cross-sectional view schematically showing a modified example of the first embodiment.

FIG. 6 is a perspective view showing a schematic structure of a semiconductor micro-analysis chip of a second embodiment.

FIG. 7 is a perspective view showing a modified example of the second embodiment.

FIG. 8 is a perspective view showing a schematic structure of a semiconductor micro-analysis chip of a third embodiment.

FIG. 9 is a cross-sectional view showing section II-II′ in FIG. 8.

FIGS. 10A to 10C are cross-sectional views showing steps of manufacturing the semiconductor micro-analysis chip of the third embodiment.

FIG. 11 is a cross-sectional view showing a modified example of the third embodiment.

FIG. 12 is a perspective view showing another modified example of the third embodiment.

FIG. 13 is a perspective view showing a schematic structure of a semiconductor micro-analysis chip of a fourth embodiment.

FIG. 14 is a cross-sectional view showing section III-III′ in FIG. 13.

FIG. 15 is a cross-sectional view showing a modified example of FIG. 14.

FIGS. 16A to 16D are cross-sectional views showing steps of manufacturing the semiconductor micro-analysis chip of the fourth embodiment.

FIG. 17 is a perspective view showing a structure of a groove portion of a first substrate used in the fourth embodiment.

FIG. 18 is a perspective view showing a structure of a groove portion of a second substrate used in the fourth embodiment.

FIGS. 19A and 19B are a plan view and a perspective view for explanation of a modified example.

FIGS. 20A and 20B are a plan view and a perspective view for explanation of another modified example.

### DETAILED DESCRIPTION

In general, according to one embodiment, there is provided an analysis chip for detection of particles in a sample liquid, comprising: a substrate; a channel provided on a surface portion of the substrate; a liquid storage portion provided on a part of the channel to store the sample liquid, holes being provided at a bottom portion of the liquid storage portion to connect the liquid storage portion and the channel; and first electrodes provided in the channel or the liquid storage portion.

A semiconductor micro-analysis chip of the embodiments will be explained hereinafter with reference to the accompanying drawings.

### First Embodiment

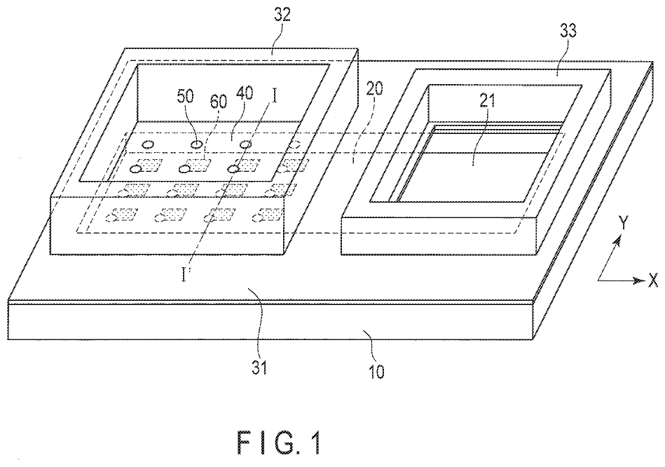

FIG. 1 is a perspective view showing a schematic structure of a semiconductor micro-analysis chip of a first embodiment.

The semiconductor micro-analysis chip of the present embodiment comprises a first microchannel 20 provided on a surface portion of a substrate 10, an insulating film 31 provided on the substrate 10 to cover an upper surface of the channel 20, micropores (holes) 50 provided in the insulating film 31 on the same end side of the channel 20, and detection electrodes (first electrodes) 60 provided on a bottom portion of the channel 20 on the same end side of the channel 20. The channel 20 is a groove shaped channel laying in the X direction of the surface portion of the substrate 10. A bank 32 formed of an insulating film is provided on the insulating film 31 on an end side of the channel 20 to surround the micropores 50, and a liquid storage portion 40 is thereby formed.

In addition, a liquid introduction reservoir 21 is provided on the other end side of the channel 20. In other words, the insulating film 31 is opened on the other end side of the channel 20 and a bank 33 formed of an insulating film is provided to surround the opened portion.

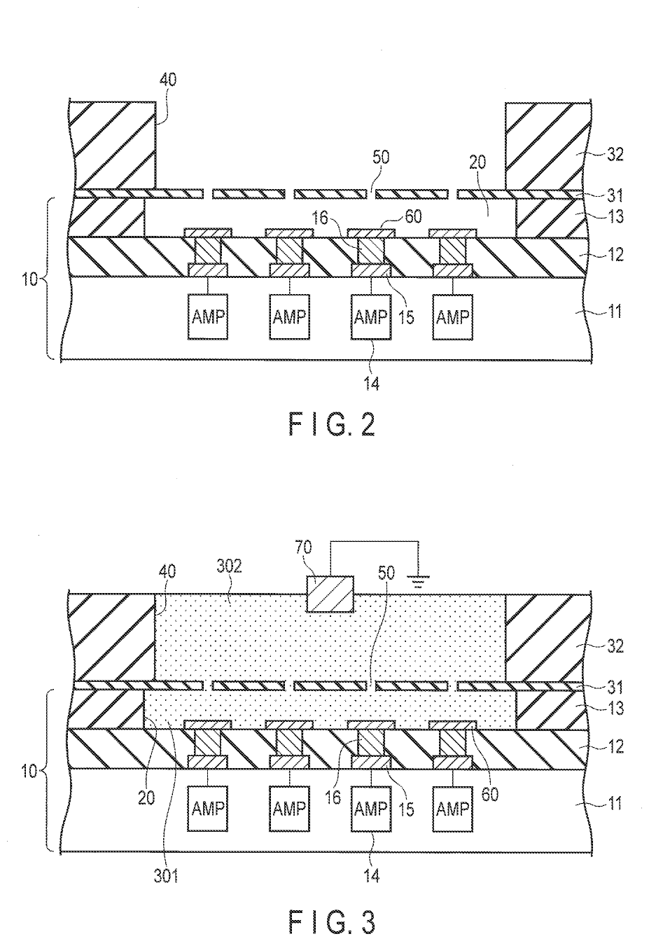

FIG. 2 is a cross-sectional view showing section I-I′ in FIG. 1, illustrating a structure of a particle detecting portion of the semiconductor micro-analysis chip shown in FIG. 1.

The end side of the channel 20 and the liquid storage portion 40 are adjacent to each other with the insulating film 31 interposed there between. The micropores 50 are provided in the insulating film 31 as particle detecting portions, and the channel 20 and the liquid storage portion 40 are spatially connected via the micropores 50. The micropores 50 are provided at regular intervals in the X direction and the Y direction. The detection electrodes 60 are provided on the bottom surface of the channel 20 to face to the respective micropores 50.

A diameter of each micropore 50 is desirably larger than detected particles. From the viewpoint of detection accuracy, the diameter of the micropore 50 is desirably, slightly larger than the size of the particles to be detected.

The substrate 10 is obtained by forming an insulating film 12 on a Si substrate 11 and further forming an insulating film 13 on the insulating film 12, and the channel 20 is produced by subjecting the insulating film 13 to selective etching and forming a groove. Then, the insulating film 31 of SiO₂ or the like is provided on the insulating film 13 to cover the channel 20. Amplifiers 14 and their contact electrodes 15 are provided on the Si substrate 11. In addition, through electrodes 16 penetrating the insulating film 12 are provided to connect with the contact electrodes 15. The detection electrodes 60 are connected to the respective contact electrodes 15 of the amplifiers 14 via the through electrodes 16.

In this structure, the channel 20 is filled with an electrolyte 301 and then a sample liquid 302 is introduced into the liquid storage portion 40 as shown in FIG. 3. The introduction of the electrolyte 301 into the channel 20 is performed by introducing the electrolyte 301 into the liquid introduction reservoir 21. The electrolyte 301 introduced into the liquid introduction reservoir 21 flows into the channel 20 by capillarity. By performing this operation in advance, air in the channel 20 is discharged through the micropores 50. When the sample liquid 302 is introduced into the liquid storage portion 40, the liquid in the channel 20 and the liquid in the liquid storage portion 40 contact without involving air bubbles in the portions of the micropores 50.

In this situation, a GND electrode (second electrode) 70 is set to be in contact with the sample liquid 302 in the liquid storage portion 40. As regards the GND electrode 70, an electrode rod may be inserted from an upper part of the liquid storage portion 40 or an electrode plate may be arranged to be in contact with the sample liquid at an upper part of the liquid storage portion 40 as shown in FIG. 3. Ag/AgCl, Au, Pt and the like can be used as the material of the GND electrode 70. In addition, a conductive film or the like may be preliminarily formed on an inner wall of the bank 32 of the liquid storage portion 40. When a potential difference is applied between the detection electrodes 60 and the GND electrode 70, an ion current flows through the micropores 50.

In a case where, for example, particles in the sample liquid 302 introduced into the liquid storage portion 40 are negatively charged, the particles contained in the sample liquid 302 are electrophoresed by an electric field generated between the detection electrodes 60 and the GND electrode 70, under the condition that the electric potential of the detection electrodes 60 is set higher than that of the GND electrode 70. Then, the particles move into the channel 20 through the micropores 50. When the particles in the liquid storage portion 40 pass through the micropores 50, the electric resistance at the micropores is increased and the ion current is varied in accordance with the size of the particles. By detecting variation in the ion current, the particles can be detected. The ion current variation detected at the detection electrodes 60 arranged just under the micropores 50 is input to the amplifiers 14 through the through electrodes 16 and the contact electrodes 15. In general, the ion current variation being small, the signals detected at the detection electrode 60 need to be amplified. Arranging the detection electrodes 60 at the bottom of the channel 20 as in the present embodiment, the shortest connection between the detection electrodes 60 and the amplifier 14 can be established via the through electrodes 16 and the contact electrodes 15. That is, it is possible to avoid signal attenuation and the like due to routing of the electrodes and the like. Therefore, the particles can be detected with high accuracy.

In the present embodiment, the particles can be thus detected only by introduction of the sample liquid and the electric observation. For this reason, high-accuracy detection of bacteria, viruses, and the like can easily be implemented. The present embodiment can therefore contribute to technical fields of prevention of spreading of epidemic diseases and food safety by application to simple detection of infectious disease pathogens, food poisoning bacteria, and the like. The present embodiment can also be applied to monitoring harmful substances such as particulate matters in a sample obtained by collecting particles suspended in the air and subjecting the particles to submerged dispersion.

In addition, by arranging a plurality of micropores 50 in the present embodiment, the frequency of passage of the particles through the micropores 50 can be efficiently increased and the detection efficiency can be enhanced. The micropores 50 through which the particles have passed can be specified by providing the detection electrodes 60 corresponding to the respective micropores 50. Furthermore, even if the particles simultaneously pass through different micropores 50, events can be detected separately.

The detection electrodes 60 are drawn to an underlayer through the insulating film 12 forming the bottom surface of the channel 20 and connected to the amplifiers 14 provided just under the insulating film 12. For this reason, the detection signals can be amplified by the amplifiers 14 without increasing noise due to routing of the electrodes, and the like. Inspection can be therefore performed with good accuracy with faint detection signals.

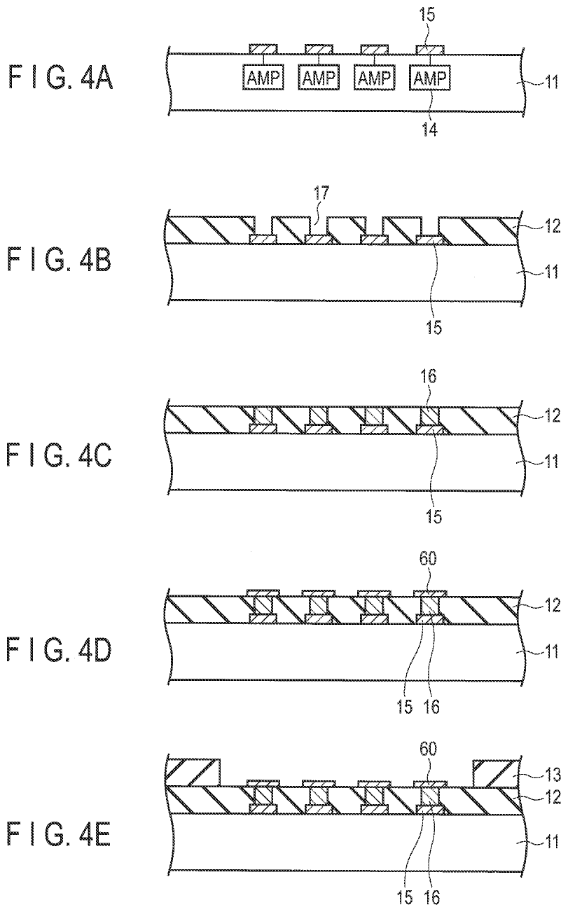

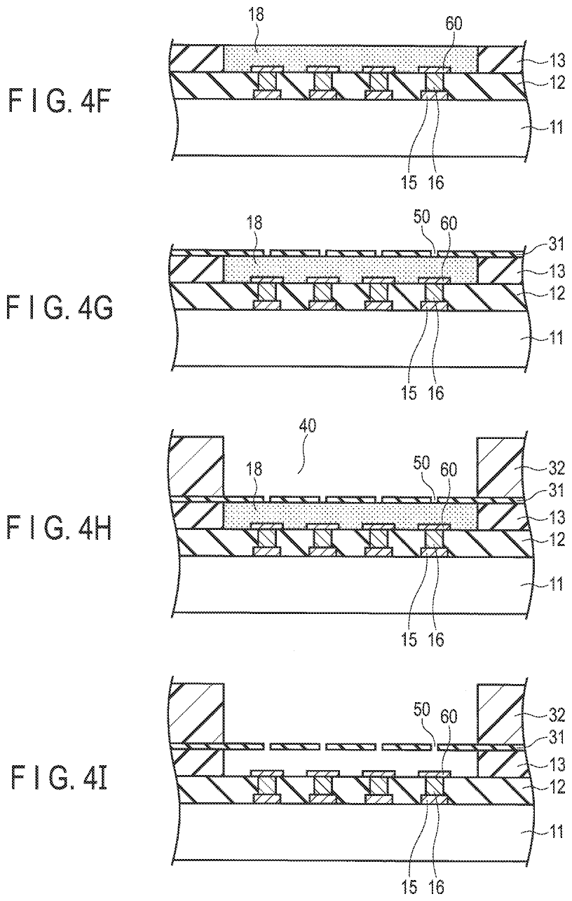

Next, a method of manufacturing the analysis chip of the present embodiment shown in FIG. 1 and FIG. 2 will be explained with reference to FIGS. 4A to 4I.

First, as shown in FIG. 4A, the Si substrate 11 on which amplifiers 14 such as CMOS circuits are formed is prepared. Contact electrodes 15 serving as connection terminals of the amplifiers 14 are provided on the Si substrate 11. Hereafter, the amplifiers 14 will be omitted in FIGS. 4B to 4I.

Next, as shown in FIG. 4B, the insulating film 12 of SiO₂ or the like is formed on the Si substrate 11 to cover the contact electrodes 15 and contact holes 17 for connection with the contact electrodes 15 are formed in the insulating film 12. The insulating film 12 is formed by, for example, chemical vapor deposition (CVD) and the like and the contact holes 17 are formed with photolithography, reactive ion etching (RIE), and the like.

Next, as shown in FIG. 4C, the through electrodes 16 are buried in the contact holes 17. More specifically, the conducting film is formed by sputtering film formation or plating to bury the contact holes 17, then the conducting film other than the film inside the contact holes 17 is removed by chemical mechanical polishing (CMP) or the like, and the through electrodes 16 are left in the contact holes 17 alone.

Next, as shown in FIG. 4D, the detection electrodes 60 are formed to be connected to the respective through electrodes 16. To form the detection electrodes 60, patterning may be performed by photolithography and RIE after forming the film of the conductive material by sputtering, vapor deposition or the like. Alternatively, the film of the conductive material may be formed after forming the resist pattern which has openings at the detection electrodes 60, and the resist pattern and an unnecessary conductive film on the resist pattern may be removed by a lift-off process. It should be noted that the through electrodes 16 and the detection electrodes 60 may be formed simultaneously. More specifically, the contact holes 17 for connection between the insulating film 12 and the contact electrodes 15 are formed and the conductive film is formed on the insulating film 12 to bury the contact holes 17. After that, the conductive film may be selectively etched in the electrode pattern.

Next, the insulating film 13 is formed on the insulating film 12 by CVD or the like to cover the detection electrodes 60. Subsequently with this, as shown in FIG. 4E, a groove portion which is to be the microchannel 20 is formed by selectively etching the insulating film 13 by photolithography and RIE.

Next, as shown in FIG. 4F, a sacrificial layer 18 is buried in the groove portion of the insulating film 13, i.e., the portion which is to be the microchannel 20. More specifically, for example, a film of a sacrificial layer material such as amorphous silicon is formed to bury the groove portion of the insulating film 13 by CVD, sputtering or the like, and subsequently, sacrificial layer 18 is left in the groove portion of the insulating film 13 alone by removing the sacrificial layer material at portions other than the inside of the groove portion of the insulating film 13 by CMP or the like. Alternatively, the insulating film 13 may be coated with a film of resin material or the like by spin coating or the like to bury the groove and portion of the insulating film 13 and the sacrificial layer 18 may be left in the groove portion of the insulating film 13 alone by CMP or etch back.

Next, as shown in FIG. 4G, an insulating film 31 is formed to have a thickness of, for example, 100 nm, on the sacrificial layer 18 and the insulating film 13 by CVD. The insulating film 31 becomes a partition which partitions the channel 20 and the liquid storage portion 40. Subsequently with this, the micropores 50 are opened in the insulating film 31 to face to the detection electrodes 60 at the portion which becomes the liquid storage portion 40. In addition, the liquid introduction reservoir 21 is opened simultaneously. The micropores 50 and the liquid introduction reservoir 21 are opened resist pattern formation by photolithography or electron beam lithography or the like and subsequent RIE or the like.

Next, as shown in FIG. 4H, the liquid storage portion 40 is formed by forming a bank 32 on one of end sides of the channel 20 to surround the micropores 50, and the liquid introduction reservoir 21 is further formed by forming a bank 33 on the other end side of the channel 20. The banks 32 and 33 are formed of, for example, a photosensitive polyimide film having a thickness of approximately 50 μm by photolithography. It should be noted that the banks 32 and 33 can be made simultaneously.

Finally, as shown in FIG. 4I, the microchannel 20 is formed by removing the sacrificial layer 18 by dry etching, wet etching or the like. The structure shown in FIG. 1 and FIG. 2 are completed in the above-explained steps.

According to the present embodiment, as described above, the analysis chip can be manufactured in a general semiconductor device manufacturing process using the Si substrate 11. Therefore, in addition to that the analysis chip of this embodiment can detect the particles with high sensitivity micromachining and mass production of the semiconductor technology can be applied to the analysis chip. For this reason, the analysis chip can be manufactured in a very small size, at low costs.

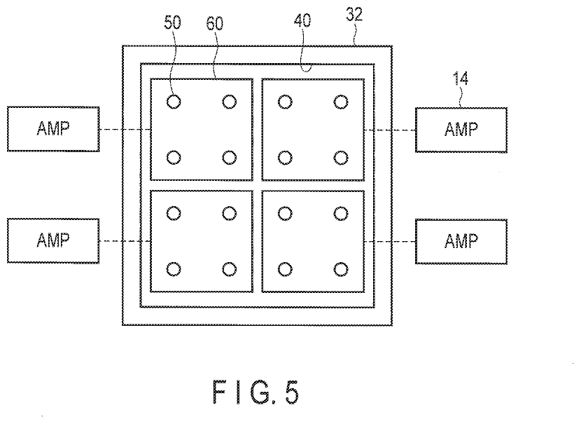

The detection electrodes 60 are provided for the respective micropores 50 in the present embodiment but one detection electrode 60 may be provided for the micropores 50. As shown in FIG. 5, for example, one detection electrode 60 is provided for four adjacent micropores 50, at a position remote from the micropores 50 in an equal distance. In this case, the current variations detected at the four micropores 50 are amplified by one amplifier 14.

In addition, the liquid introduced into the channel 20 is not limited to the electrolyte but the channel 20 may be filled with the sample liquid.

### Second Embodiment

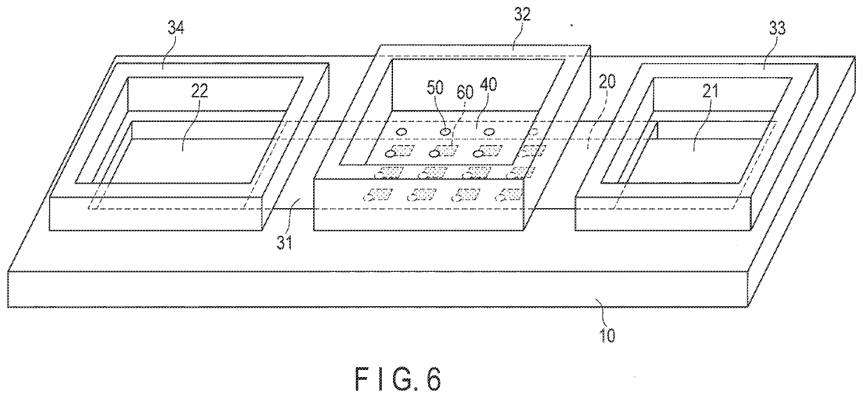

FIG. 6 is a perspective view showing a schematic structure of a semiconductor micro-analysis chip of a second embodiment. Elements like or similar to those shown in FIG. 1 are denoted by the same reference numbers and their detailed explanations are omitted. An insulating film 31 is illustrated simply.

The present embodiment is different from the first embodiment with respect to a feature that a liquid discharge reservoir 22 is provided in a microchannel 20. In other words, the liquid discharge reservoir 22 is produced by opening the insulating film 31 on one of end sides of the channel 20 and providing a bank 34 so as to surround the opening portion. A liquid introduction reservoir 21 is provided on the other end side of the channel 20, similarly to the first embodiment. Furthermore, a liquid storage portion 40 is provided on a central portion of the channel 20.

In such a structure, a sample liquid or an electrolyte can be introduced from the liquid introduction reservoir 21 and discharged from the liquid discharge reservoir 22, and a smooth flow of the sample liquid or the electrolyte in the channel 20 can be implemented. A risk of taking in air bubbles through micropores 50 when the sample liquid is dropped into the liquid storage portion 40 can be reduced. In addition, if particles moving from the liquid storage portion 40 into the channel 20 through the micropores 50 are retained in the channel 20, the particles may become a cause of noise in an ion current. However, the particles can be discharged efficiently by implementing a smooth flow of the electrolyte in the channel 20 by the above-described structure of the present embodiment. In other words, high-accuracy measurement reducing noise can be performed. Therefore, according to the present embodiment, in addition that the same advantages as those of the first embodiment can be naturally obtained, the reliability can be increased and the accuracy can be made higher by a smooth flow of the electrolyte in the channel 20.

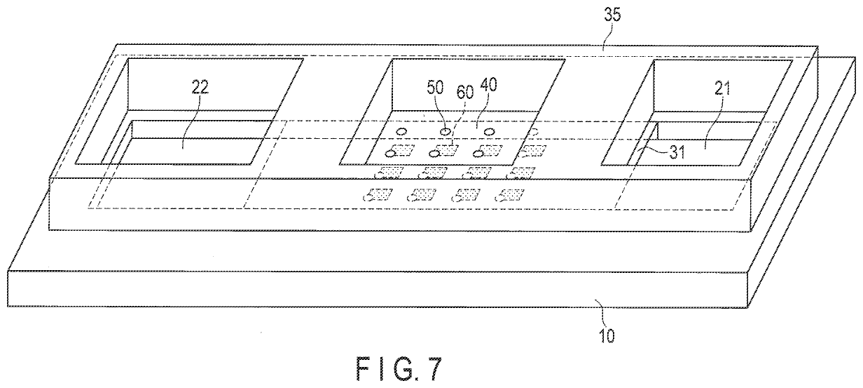

In the present embodiment, banks 33 and 34 for the respective reservoirs 21 and 22, and a bank 32 for the liquid storage portion 40 are formed separately, but may be formed simultaneously. As shown in FIG. 7, for example, openings corresponding to the liquid storage portion 40 and the reservoirs 21 and 22 may be formed in the same insulating film 35.

### Third Embodiment

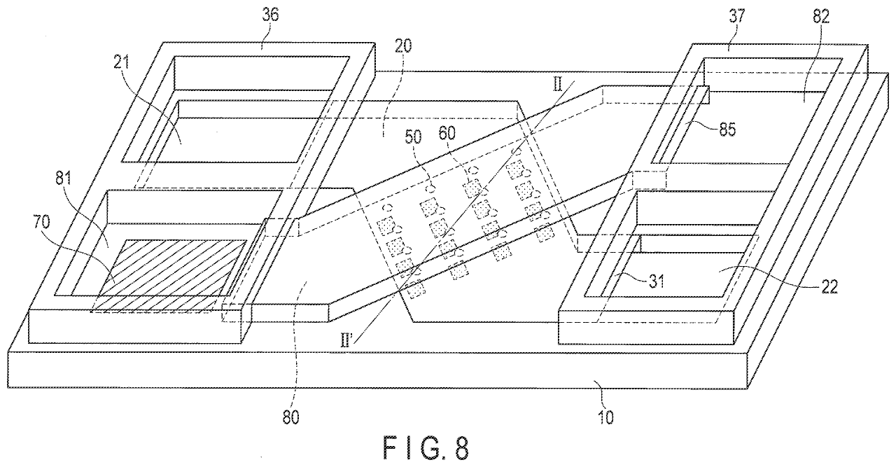

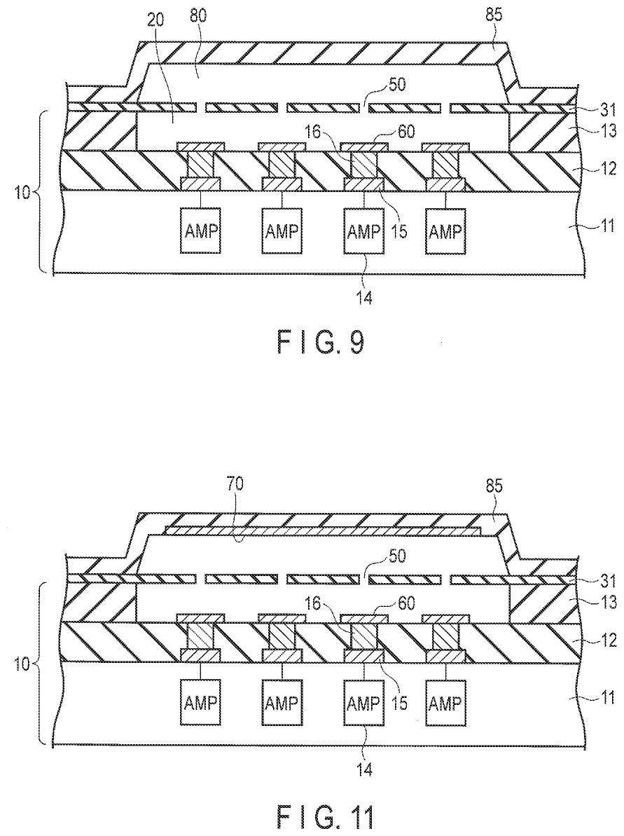

FIG. 8 is a perspective view showing a schematic structure of a semiconductor micro-analysis chip of a third embodiment. FIG. 9 is a cross-sectional view showing section II-II′ in FIG. 8, illustrating a structure of a particle detecting portion of the semiconductor micro-analysis chip shown in FIG. 8. Elements like or similar to those shown in FIG. 1 and FIG. 2 are denoted by the same reference numbers and their detailed explanations are omitted.

The semiconductor micro-analysis chip of the present embodiment comprises a first microchannel (first channel) 20 provided on a surface portion of a substrate 10, an insulating film 31 covering an upper surface of the channel 20, a second microchannel (second channel) 80 provided on the insulating film 31 so as to make overhead crossing with the channel 20, micropores 50 provided in the insulating film 31 at the portion of the overhead crossing of the channels 20 and 80, detection electrodes (first electrodes) 60 provided at the bottom of the channel 20, and a GND electrode (second electrode) 70 provided on a part of the channel 80.

The channel 20 and the channel 80 make overhead crossing at a central portion of the surface of the substrate 10. The channel 20 is produced by processing the surface portion of the substrate 10 so as to be in a groove shape by selective etching. The channel 80 is formed in tunnel shape obtained by surrounding a space which is to be a channel by an insulating film 85.

The liquid introduction reservoir 21 to introduce the sample liquid or the electrolyte is provided on one of end sides of the channel 20, and the liquid discharge reservoir 22 to discharge the sample liquid or the electrolyte is provided on the other end side of the channel 20. The reservoirs 21 and 22 are produced by opening the insulating film 31 on one end side and the other end side of the channel 20 and providing banks 36 and 37 so as to surround the opened portions.

A liquid introduction reservoir 81 to introduce the sample liquid or the electrolyte is provided on one of end sides of the channel 80, and a liquid discharge reservoir 82 to discharge the sample liquid or the electrolyte is provided on the other end side of the channel 80. The liquid introduction reservoir 81 is produced by providing the bank 36 so as to surround a space connecting to one of ends of the channel 80. The liquid discharge reservoir 82 is produced by providing the bank 37 so as to surround a space connecting to the other end of the channel 80. In other words, the bank 36 is common to the liquid introduction reservoirs 21 and 81 and the bank 37 is common to the liquid discharge reservoirs 22 and 82. In addition, the GND electrode 70 is provided on the liquid introduction reservoir 81.

As shown in FIG. 9, the substrate 10 is obtained by forming an insulating film 12 on a Si substrate 11 and further forming an insulating film 13 on the insulating film 12, and the channel 20 is provided by subjecting the insulating film 13 to selective etching and forming a groove. Then, the insulating film 31 of SiO₂ or the like is provided on the insulating film 13 to cover the channel 20. The insulating film 85 is formed on the insulating film 13 so as to cover the space to be the channel 80. The channel 20 and the channel 80 have a structure in which the channels are deposited to sandwich the insulating film 31 at the intersection portion. In addition, the detection electrodes 60 corresponding to the respective micropores 50 are provided on the bottom surface of the channel 20. In the present embodiment, the detection electrodes 60 are arranged just under the respective micropores 50. The detection electrodes 60 do not need to be provided for each the micropores 50, but one detection electrode 60 may be provided for some adjacent micropores 50.

Amplifiers 14 and their contact electrodes 15 are provided at corresponding positions just under the detection electrodes 60, in the Si substrate 11. In addition, through electrodes 16 penetrating the insulating film 12 are provided to be connected to the contact electrodes 15, and the through electrodes 16 are connected to the detection electrodes 60.

In this structure, when the sample liquid containing the particles dispersed is introduced from the liquid introduction reservoir 81 of the channel 80, the sample liquid flows in the channel 80. The channel 20 is preliminarily filled with the electrolyte in advance. The liquid in the channel 80 and the liquid in the channel 20 thereby contact via the micropores 50. The liquid introduced into the channel 20 is not limited to the electrolyte but the channel 20 may be filled with the sample liquid.

When a potential difference is made between the detection electrodes 60 and the GND electrode 70 in this state, an ion current flows through the micropores 50. In addition, setting the electric potential of the detection electrodes 60 to be higher than the electric potential of the GND electrode 70, the particles in the sample liquid introduced into the liquid introduction portion 81 are electrophoresed to move into the channel 20 through the micropores 50 by an electric field generated between the detection electrodes 60 and the GND electrode 70 in a case where the particles are negatively charged. When the particles flowing in the channel 80 pass through the micropores 50, the ion current is varied in accordance with the size of the particles. By detecting the ion current variation, the particles can be detected. The ion current variation is input from the detection electrodes 60 arranged just under the micropores 50 to the amplifiers 14 through the through electrodes 16 and the contact electrodes 15. The particles can be therefore detected with high accuracy by amplifying the variation in the ion current value by the amplifiers 14.

In the case that the particles in the sample liquid are positively charged, the sample liquid may be introduced into the channel 20, the electrolyte may be introduced into the channel 80, and the electric potential of the detection electrodes 60 may be set to be higher than the electric potential of the GND electrode 70. In this case, the particles in the sample liquid move from the channel 20 to the channel 80 through the micropores 50. When the particles flowing in the channel 20 pass through the micropores 50, the ion current is varied in accordance with the size of the particles. Alternatively, in the case that the particles in the sample liquid are positively charged, the sample liquid may be introduced into the channel 80, the electrolyte may be introduced into the channel 20, and the electric potential of the detection electrodes 60 may be set to be lower than the electric potential of the GND electrode 70. Thus, in the structure of the present embodiment, the positive or negative charge of the particles and the electric potentials of the detection electrodes 60 and the GND electrode 70 can be combined freely in accordance with the purposes.

In the present embodiment, the particles can be thus detected by introduction of the sample liquid and the electric observation alone. In addition, the detection efficiency can be enhanced by arranging a plurality of micropores 50, because the frequency of passage of the particles through the micropores 50 can be efficiently increased. The same advantages as those of the first embodiment can be therefore obtained.

In addition, the present embodiment also has an advantage that the intersecting portion of the channels 20 and 80 can be smoothly filled with the sample liquid and the electrolyte since two channels 20 and 80 are used and the liquid is allowed to flow in each of the channels.

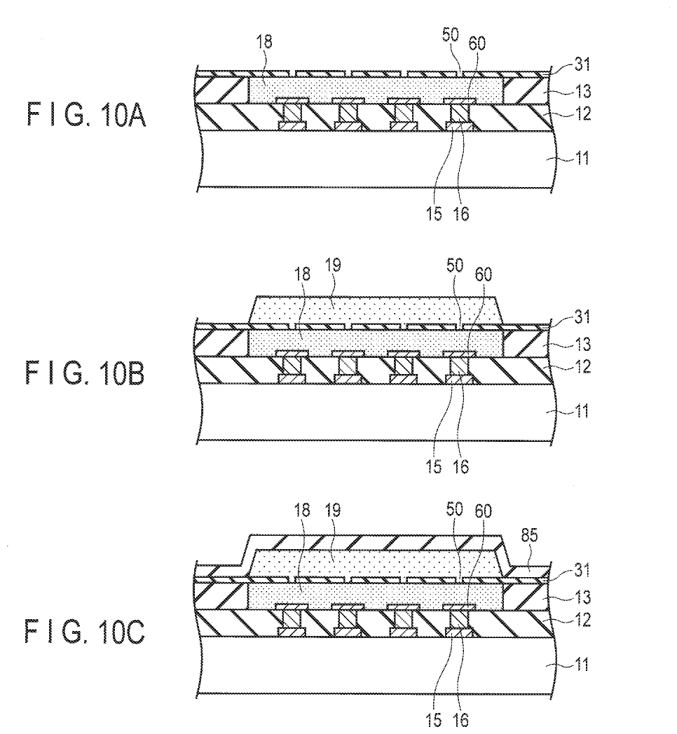

FIGS. 10A to 10C are cross-sectional views showing steps of manufacturing the semiconductor micro-analysis chip of the present embodiment. The steps are the same as those of the first embodiment shown in FIGS. 4A to 4G until the formation of the insulating film 13 and subsequent opening of the micropores 50. FIG. 10A is correspond to FIG. 4G.

Next, as shown in FIG. 10B, a second sacrificial layer 19 is formed on the entire surface and subsequently the second sacrificial layer 19 is subjected to selective etching to become a shape of the channel 80. As the material of the sacrificial layer 19, for example, an amorphous silicon CVD film or the like is used and processing of the sacrificial layer 19 is performed by photolithography, RIE and the like.

Next, as shown in FIG. 10C, an insulating film 85 of SiO₂ or the like is formed to cover the sacrificial layer 19. More specifically, the insulating film 85 is formed on an entire surface by CVD and then the insulating film 85 at portions corresponding to a liquid introduction reservoir 21 and a liquid discharge reservoir 22 is removed by photolithography and RIE. Subsequently with this, the banks 36 and 37 are formed, the GND electrode 70 is further formed, the sacrificial layers 18 and 19 are finally removed by dry etching or the like, and the channels 20 and 80 are thereby formed. The semiconductor micro-analysis chip of the present embodiment shown in FIG. 8 and FIG. 9 is thereby completed.

According to the present embodiment, as described above, the analysis chip can be manufactured in a general semiconductor device manufacturing process using the Si substrate 11. Therefore, in addition to that the analysis chip of this embodiment can detect the particles with high sensitivity, micromachining and mass production of the semiconductor technology can be applied to the analysis chip. The same advantages as those of the first embodiment can be therefore obtained.

The GND electrode 70 does not need to be formed on the liquid introduction reservoir 81 but may be formed on the liquid discharge reservoir 82. The GND electrode 70 may be provided at a position in contact with the sample liquid or the electrolyte in the channel 80. As shown in the cross-sectional view of FIG. 11, for example, the GND electrode 70 may be provided on a lower surface of the insulating film 85 of a particle detecting portion. In this case, since the GND electrode 70 and the detection electrodes 60 become closer, the sensitivity of detection of the particles can be made further higher.

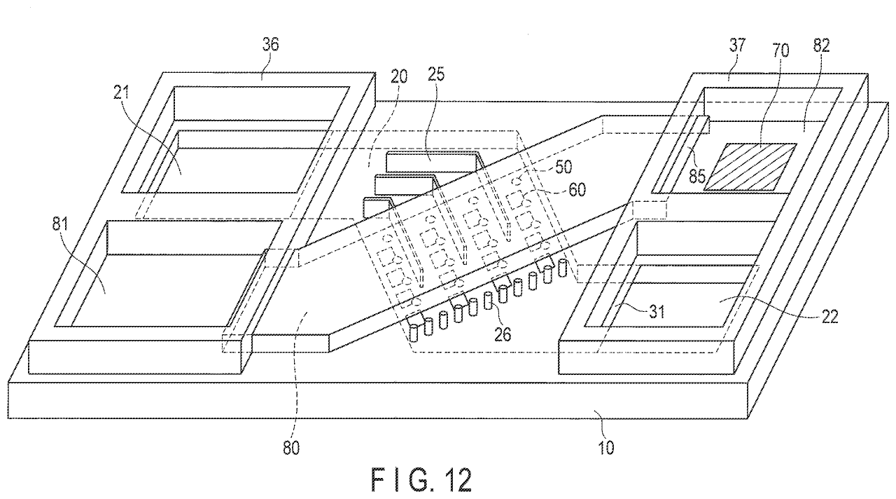

In addition, as shown in FIG. 12, division walls 25 to divide the channel 20 into plural channels may be provided at the intersecting portion from an upstream side of the channel 20, to enable the particles to move smoothly in the channel 20. The division walls 25 are provided along the channel direction and make a width of each of the divided channels smaller.

Furthermore, a particle trap mechanism formed of micropillars 26 may be provided on a downstream side of the channel 20. The micropillars 26 are aligned at intervals slightly smaller than a diameter of the particles to be detected.

The division walls 25 are produced by leaving the insulating film 13 in a plate shape with a line-shaped mask when the insulating film 13 is process in a groove shape. Moreover, the micropillars 26 are produced by leaving the insulating film 13 in a pillar shape with a circular mask when the insulating film 13 is process in a groove shape.

### Fourth Embodiment

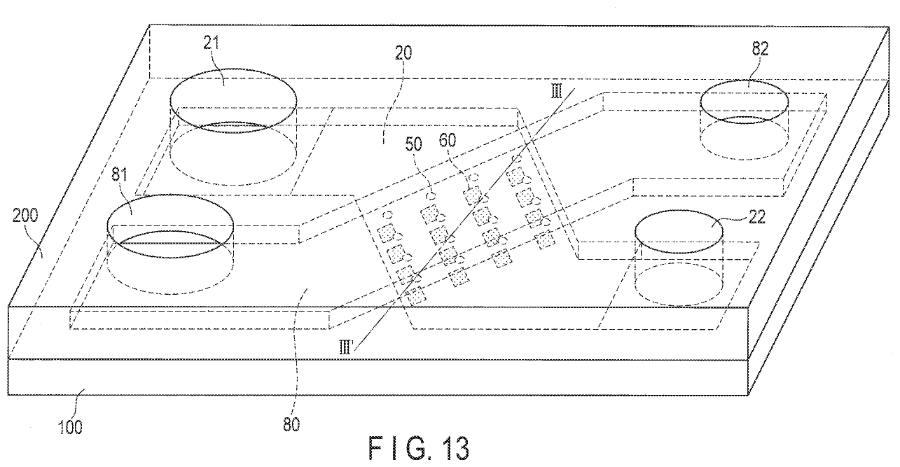

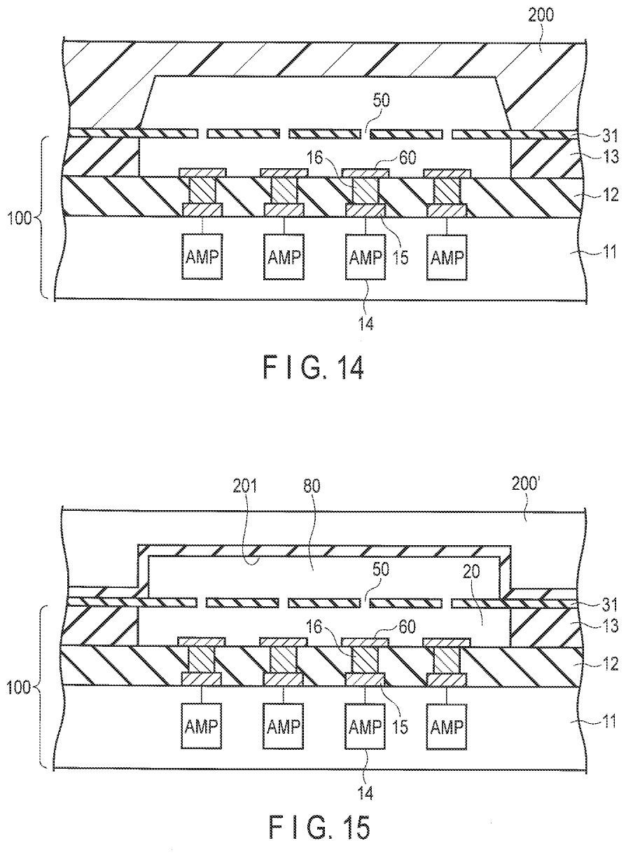

FIG. 13 is a perspective view showing a schematic structure of a semiconductor micro-analysis chip of a fourth embodiment. FIG. 14 is a cross-sectional view showing section III-III′ in FIG. 13, illustrating a structure of a particle detecting portion of the semiconductor micro-analysis chip shown in FIG. 13. Elements like or similar to those shown in FIG. 8 and FIG. 9 are denoted by the same reference numbers and their detailed explanations are omitted.

The basic structure of the present embodiment is the same as that of the third embodiment, in the viewpoint that the channels 20 and 80 make overhead crossing. The present embodiment is different from the third embodiment with respect to a feature that microchannels 20 and 80 are produced by bonding two substrates 100 and 200 to each other.

The first microchannel 20 is provided on a surface portion of the first substrate 100. The first substrate 100 is substantially the same as the substrate 10 of the first embodiment. More specifically, insulating films 12 and 13, amplifier 14, contact electrodes 15, through electrodes 16, detection electrodes 60 and the like are formed on a Si substrate 11.

The material of the second substrate 200 is, for example, plastic or quartz, and the microchannel 80 is provided by forming a groove on its lower surface. Furthermore, openings for formation of reservoirs are provided in the second substrate 200. Two channels 20 and 80 make overhead crossing by bonding the substrates 100 and 200 interposing an insulating film 31.

If a Si substrate is used as a second substrate 200′, the channel surface is desirably subjected to thermal oxidation and an oxidized film 201 is formed as shown in a cross-sectional view of FIG. 15, to ensure hydrophilicity and insulate the electrolyte and the Si substrate from each other.

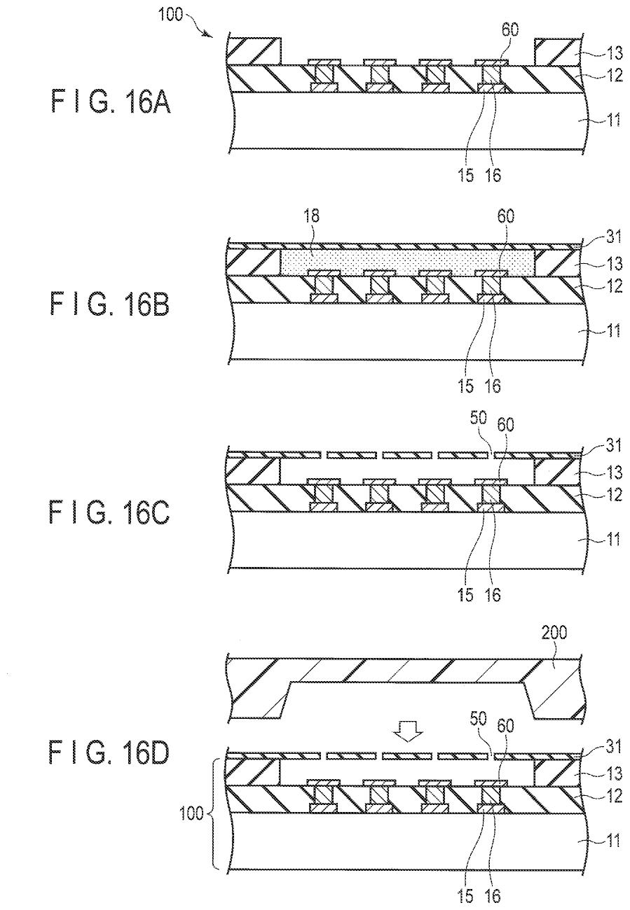

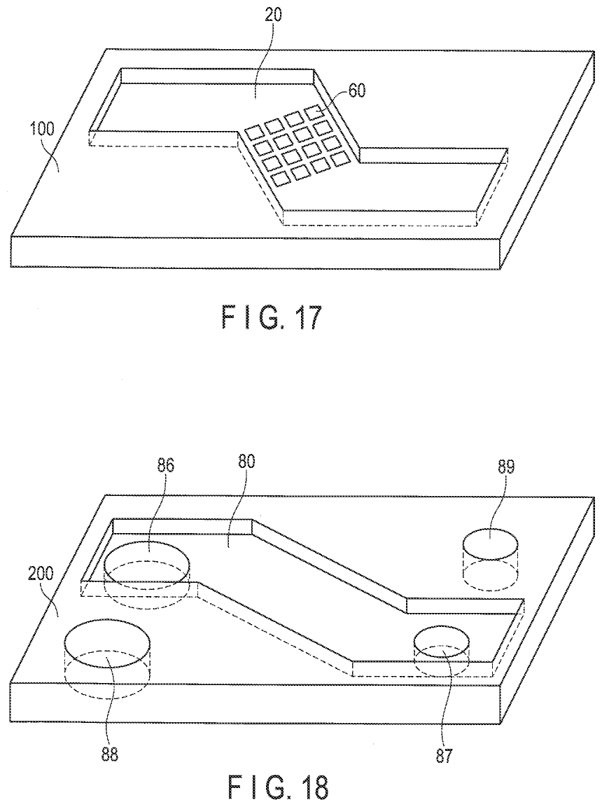

FIGS. 16A to 16D are cross-sectional views showing steps of manufacturing the semiconductor micro-analysis chip of the present embodiment. The steps are the same as those of the first embodiment shown in FIGS. 4A to 4E until the groove for the first microchannel 20 is formed on the first substrate 100. FIG. 16A is similar to FIG. 4E and a perspective view of FIG. 16A is FIG. 17.

Next, as shown in FIG. 16B, a first sacrificial layer 18 is formed in the groove portion of the insulating film 13 and the surface is flattened. Subsequently with this, a thin insulating film 31 is formed on the sacrificial layer 18 and the insulating film 13. The insulating film 31 becomes a partition film which partitions the channels 20 and 80.

Next, as shown in FIG. 16C, the micropores 50 are opened in the insulating film 31 at the portion which becomes the overhead crossing portion of the channels 20 and 80. Subsequently with this, the channel 20 is formed by removing the sacrificial layer 18 by dry etching or the like.

On the other hand, as shown in FIG. 18, a groove for the channel 80 is formed on the surface portion of the second substrate 200. The channel 80 may be formed by injection molding or the like when a resin material is used as the substrate 200, and the channel 80 may be formed by photolithography and wet etching and the like when a glass substrate is used as the substrate 200. Furthermore, an opening 86 for the liquid introduction reservoir 81 is formed on one of end sides of the groove, and an opening 87 for the liquid discharge reservoir 82 is formed on the other end side of the groove. In addition, an opening 88 for the liquid introduction reservoir 21 and an opening 89 for the liquid discharge reservoir 22 are formed on the second substrate 200, at portions which overlap the channel 20 when the first substrate 100 and the second substrate 200 are overlaid.

Then, by bonding the substrates 100 and 200 interposing the insulating film 31 as shown in FIG. 16D, the structure of overhead crossing of the channels 20 and 80 can be implemented as shown in FIG. 13.

In the present embodiment, the insulating film 31 is provided on the first substrate 100 side before bonding the substrates 100 and 200 but the insulating film 31 may be provided on the second substrate 200 side. In addition, the sacrificial layer 18 may be removed after bonding the substrates 100 and 200 via the holes 86 to 89.

The final structure of the present embodiment is substantially the same as that of the third embodiment and the same advantages as those of the third embodiment can be therefore obtained. In addition to this, the manufacturing process can be simplified and the manufacturing costs can be reduced since the present embodiment can be implemented by bonding the substrates 100 and 200 to each other.

### Modified Example

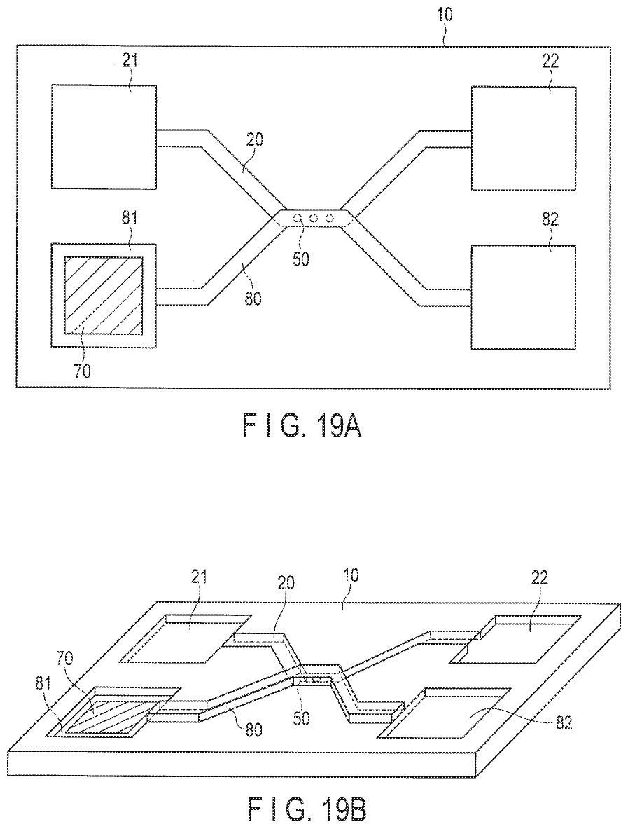

The invention is not limited to the above-described embodiments. The first channel and the second channel in the third and fourth embodiments do not need to intersect but may be partially adjacent to each other as shown in a plan view of FIG. 19A and a perspective view of FIG. 19B. In this case, micropores 50 are formed at the adjacent portion (stacking portion) of the first channel 20 in the groove shape and the second channel 80 in the insulating film tunnel shape.

In this structure, too, the particles can be detected by introduction of the sample liquid and the electric observation alone and, furthermore, the frequency of passage of the fine particles through the micropores 50 can be efficiently increased by arranging a plurality of micropores 50. The same advantages as those of the third embodiment can be therefore obtained.

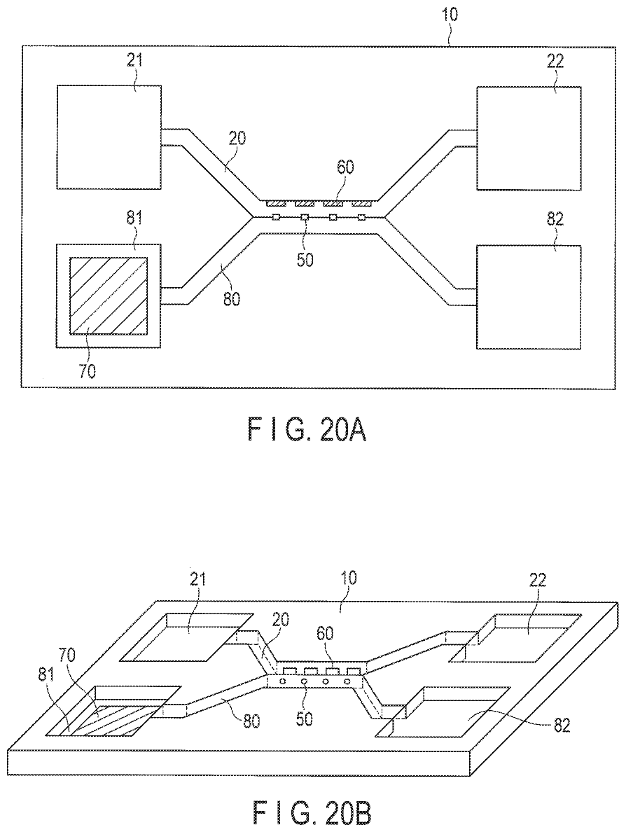

In addition, both the channels 20 and 80 may be channels in the groove shape as shown in a plan view of FIG. 20A and a perspective view of FIG. 20B. In other words, the channel 20 is produced by processing the surface portion of the substrate 10 so as to be in a groove shape by selective etching, similarly to the third embodiment, and the channel 80 is also produced by processing the surface portion of the substrate 10 so as to be in a groove shape by selective etching, similarly to the channel 20, unlike the third embodiment. In addition, the channels 20 and 80 do not intersect but are partially adjacent to each other. Then, a plurality of micropores 50 are provided in a partition film at the adjacent portion of the channels 20 and 80.

The micropores 50 may be shape in a circle or may be formed in a slit shape at the adjacent portion of the channels 20 and 80. Furthermore, the detection electrodes 60 are formed on a side wall of the channel 20 so as to be opposed to the micropores 50, but may be formed on the bottom surface of the channel 20.

In this structure, too, the particles can be detected by introduction of the sample liquid and the electric observation alone and, furthermore, the frequency of passage of the fine particles through the micropores 50 can be efficiently increased by arranging a plurality of micropores 50. The same advantages as those of the third embodiment can be therefore obtained.

In addition, the first electrodes are arranged on the first channel side and the second electrode is arranged on the second channel side or the liquid storage side in the embodiments, but these electrodes may be arranged on opposite sides. Moreover, the number of holes and the number of detection electrodes can be arbitrarily changed in accordance with specifications.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An analysis chip for detection of particles in a sample liquid, comprising:
a substrate;
a first channel provided on a surface portion of the substrate;
a second channel provided on or above the surface portion of the substrate, and being partially adjacent to the first channel;
a partition provided at a portion where the first and second channels are adjacent to each other, and including one or more hole portions through which the first and second channels are connected; and
first electrodes provided in the first or second channels, and corresponding to the hole portions.

2. The analysis chip of claim 1, wherein each of the first electrodes is provided for one or a predetermined number of hole portions, at the bottom portion of the first channel.

3. The analysis chip of claim 2, further comprising a second electrode provided in the second channel.

4. The analysis chip of claim 2, wherein the substrate includes a semiconductor substrate, an insulating film provided on the semiconductor substrate, and an amplifier provided at the semiconductor substrate and connected to the first electrodes.

5. The analysis chip of claim 4, wherein the first electrodes are provided on the insulating film and connected to the amplifier via a through electrode penetrating the insulating film.

6. The analysis chip of claim 3, wherein
each of the first and second channels includes a liquid introduction reservoir for introduction of the liquid and a liquid discharge reservoir for discharge of the liquid, and
the second electrode is provided in the liquid introduction reservoir and the liquid discharge reservoir of the second channel.

7. The analysis chip of claim 1, further comprising a particle trap structure arranged in the first channel.

8. The analysis chip of claim 1, wherein the first channel is a channel of a groove shape provided on the surface portion of the substrate, the second channel is a channel of an insulating film tunnel shape provided on the substrate, a part of the first channel and a part of the second channel intersect, and the hole portions are provide at the intersection portion.
